SEQUENCE LISTING (1 sequence)

SEQ ID NO: 1 - protein, 26 aa, Cannabis Sativa L.
  Hemp beta-expansin signal peptide
MQLVRSVCFGALTLLLVFKYSLLVDA

(19) United States
(12) Patent Application Publication
(10) Pub. No.: US 2019/0119693 A1
(43) Pub. Date: Apr. 25, 2019

(54) Genetically Engineered Plant Fibres Presenting Enhanced Surface Properties

(71) Applicant: Luxembourg Institute Of Science And Technology LIST), Esch/Alzette (LU)

(72) Inventor: Gea Guerriero, Esch/Alzette (LU)

(21) Appl. No.: 15/759,209

(22) PCT Filed: Sep. 5, 2016
(86) PCT No.: PCT/EP2016/070797

§ 371 Date: Mar. 10, 2018

(30) Foreign Application Priority Data
Sep. 11, 2015 (LU) 92825

(51) Int. Cl. C12N 15/82

(52) U.S. Cl. CPC C12N 15/8226; C12N 15/8242

(57) ABSTRACT

A genetic engineering process of fibrous plant comprising bast fibres. The process comprises the steps of (a) identification of the bast fibre promoter and (b) amplification of the bast fibre promoter. The process is remarkable in that it further comprises the step (c) of preparing a gene cassette by fusing the bast fibre promoter with at least one gene coding for a surface-active protein. Additionally, the fibrous plant obtained by the genetic engineering process. The fibrous plant is remarkable in that it comprises surface-active proteins.

### CROSS-REFERENCE TO RELATED APPLICATIONS

The present invention is the US national stage under 35 U.S.C. § 371 of International Application No. PCT/EP2016/070797, which was filed on Sep. 5, 2016, and which claims the priority of application LU 92825 filed on Sep. 11, 2015, the content of which (text, drawings and claims) are incorporated here by reference in its entirety.

### FIELD

The invention is directed to the genetic engineering of plant fibres, notably fibre crops, in order to modify the fibre properties, in particular the surface properties such as hydrophobicity.

### BACKGROUND

The use of plant fibres, in particular fibre crops, for the reinforcement of biocomposite is important. An advantage for using plant fibres is that they are renewable, cheap and they raise no health-related issues. However, plant fibres are hygroscopic. Particularly, it is known that when the biocomposites absorb moisture, the plant fibres swell and their subsequent shrinking triggers the formation of cracks. The plant fibres do not bind well with the different resins that are used in this field because of their hydrophilic nature.

An advantage of the fibre crops is that they produce bast fibres. The bast fibres are defined as extraxylary sclerenchymatous elements obtained from the stem cortex of various plants and can be used as reinforcements for polymeric materials. Those fibres are composed primarily of cellulose which potentially has a Young's modulus of about 140 GPa (a value comparable with manmade aramid (Kevlar/Twaron) fibres (Summerscales J. et al, Composites: Part A, 2010, 41, 1329-1335). The plants that are currently attracting most interest are flax, hemp, jute (Corchorus), kenaf, sisal, ramie, cotton and nettle. In the automotive industry, it is particularly known that plastics made of hemp fibres are used in the manufacture of different components of the car because those fibres are extremely light and extremely resistant to high pressures. Furthermore, those fibre crops, differently from glass fibres, do not present health-related issue.

In order to make the plant fibres hydrophobic, and subsequently to address the above-mentioned drawback of swelling and shrinking depending on the moisture and the poor binding with the resins, different physico-chemical treatments can be performed. But, unfortunately, those can lead to the alteration of the fibre properties and/or of the fibre morphology.

Amphipathic proteins are known to self-assemble into rodlets to coat the aerial hyphae of some filamentous bacteria and fungi with a hydrophobic sheath. Chaplins, rodlins, streptofactins and hydrophobins are examples of those particular proteins. Those types of proteins, secreted by the filamentous bacteria and fungi, lower the water surface tension to allow aerial growth and cover aerial structures, rendering them hydrophobic.

In the paper entitled “LuFLA1_{PRO} and LuBGAL1_{PRO} promote gene expression in the phloem fibres of flax (Linum usitatissimum)” (Hobson H., et al, Plant Cell Rep., 2013, 32, 517-528), the authors described the identification and the isolation of flax gene promoters to direct transgene expression in developing and maturing phloem fibres of flax.

European patent application published EP 2 631 296 A1 relates to a method to produce hydrophobins in plants and microbes. The plants are used as “green” factories for the production of those industrially-relevant proteins. Transgenic plants, transgenic seeds and expression cassette are indeed produced by the described method. The proteins are isolated from the leaves or from the seeds of the plant.

International patent application published WO 2011/089021 A1 describes cotton fibres comprising positively charged polysaccharides such as chitin and chitosan. The plant secondary cell walls are thus modified.

### SUMMARY

The invention has for technical problem to provide plant fibres that present surface properties, in particular hydrophobicity, improving the biocomposite properties and without altering the intrinsic properties and morphology of the plant fibres.

The first object of the invention is directed to a genetic engineering process of fibrous plant comprising bast fibres. The process comprises the steps of (a) identification of the bast fibre promoter; and (b) amplification of the bast fibre promoter. The process is remarkable in that it further comprises the step (c) of preparing a gene cassette by fusing the bast fibre promoter with at least one gene coding for a surface-active protein.

In various embodiments, the step (c) further comprises the incorporation of the gene of the β-expansin signal peptide to the gene cassette.

In various embodiments, the β-expansin signal peptide is SEQ ID NO:1.

In various embodiments, the process further comprises the step (d) of cloning the gene cassette in a first vector, in various instances the pENTR™/D-TOPO® vector.

In various embodiments, the process further comprises the step (e) of recombining the first vector, in various instances the pENTR™/D-TOPO® vector, into a second vector, in various instances the pEarleyGate 302 vector.

In various embodiments, the step (a) is performed by formation of complementary deoxyribonucleic acid libraries (cDNA libraries) and subsequent high-throughput sequencing (RNA-Seq), the formation and sequencing being in various instances performed by using Illumina® Sequencing Technology.

In various embodiments, the step (a) is performed in the above snap-point, (ASP) part and/or in the below snap-point (BSP) part of the stem of the fibrous plant.

In various embodiments, the step (b) is performed by means of polymerase chain reaction (PCR).

In various embodiments, the surface-active protein is selected from the group of hydrophobins, chaplins, rodlins, and streptofactins, in various instances hydrophobins.

In various embodiments, the fibrous plant is selected from the group of flax, hemp, jute, kenaf, ramie, sisal, cotton and nettle, in various instances hemp.

In various embodiments, the process is performed via Agrobacterium tumefaciens GV3101.

The second object of the invention is directed to a fibrous plant obtained by the genetic engineering process in accordance with the first object of the invention. The fibrous plant is remarkable in that the fibrous plant comprises surface-active proteins.

In various embodiments, the surface-active proteins are selected from the group of hydrophobins, chaplins, rodlins and streptofactins, in various instances hydrophobins.

In various embodiments, the fibrous plant is selected from the group of flax, hemp, jute, kenaf, ramie, sisal, cotton and nettle, in various instances hemp.

In various embodiments, the surface of the fibrous plant is hydrophobic.

The invention is particularly interesting in that the genetically modified plants will produce those surface-active proteins, therefore modifying their intrinsic properties, in particular hydrophobicity. As no chemical treatments are involved, no alteration in the fibre morphology will be noticed. Those plants, more specifically fibre crops, even more specifically hemp, might be used in different implementations, such as the manufacture of waterproof paper, self-cleaning (e.g. via lotus-effect), and/or waterproof textile. Other implementations in the biocomposite industry and/or in the green biotechnology will probably follow on from those genetically engineered plants. The automotive industry will also use those fibres with enhanced surface properties.

### DRAWINGS

FIG. 1 exemplarily illustrates the PCR process between the bast-fibre promoter and the gene for surface-active protein, in accordance with various embodiments of the invention.

FIG. 2 exemplarily illustrates the PCR process between the bast-fibre promoter, the gene for surface-active protein and the gene for the β-expansin signal peptide, in accordance with various embodiments of the invention.

FIG. 3 exemplarily depicts the pEarleyGate 302 plasmid (from http://www.snapgene.com/resources/plasmid_files/plant_vectors/pEarleyGete_3 02/).

### DETAILED DESCRIPTION

In order to provide plant fibres that present hydrophobic surface properties without altering the intrinsic mechanical properties and morphology of the plant fibres, the technique of genetic engineering will be employed. The goal is to create transgenic fibre crops. The transgenic fibre crops are thus capable to secrete those surface-active proteins, in particular hydrophobins but also chaplins, rodlins and streptofactins.

The fibre crops are selected from the group consisting of flax, hemp, jute, kenaf, sisal, ramie cotton and nettle. Hemp can be used as plant of choice, in the light of its wide industrial applications.

To perform the genetic engineering of the fibre crops and, more specifically to achieve the expression of the surface-active proteins in bast fibres, the promoter of the gene expressed in the bast fibres of the fibre crops must be identified. It will therefore be necessary to identify marker genes for bast fibre thickening, in order to use their promoters to drive expression of the transgene.

It is desirable to express the foreign genes during fibre thickening, to avoid possible interference during the elongation phase of the fibre cells. The preferential expression of the genes during this stage will guarantee that the fibres can carry out water/solute exchanges necessary for turgor pressure maintenance during active elongation.

RNA-Sequencing (RNA-Seq), also called whole transcriptome shotgun sequencing (WTSS), will then be carried out on fibres separated from top and bottom internodes (plants aged 1 month) on the fibre crops, in particular hemp.

A stem of fibre crops can indeed be divided into two zones separated by the snap-point. A first zone is the ASP part, namely the Above Snap-Point part. A second zone is the BSP part, namely the Below Snap-Point.

The separation between top and bottom will enable the identification of genes enriched in two different stages of fibre formation, i.e. elongation and thickening, respectively. RNA will be separated from the collected bast fibres (three biological replicates, each consisting of a pool of 8-10 plants showing homogeneous height, stem thickness and number of internodes).

The promoter of the gene of the bast fibres is best marked in the BSP part, because the bottom of the hemp undergoes girth increase (i.e. secondary growth), while the top of the hemp elongates rapidly.

In fact, it is preferable to choose genes expressed in the bast fibres coming from the BSP part of the hemp because one does not want to interfere with the elongation of the fibres.

cDNA libraries are thus prepared using the Illumina sequencing technology.

After quality control and normalization, the pooled libraries will be processed using an Illumina sequencing platform (MiSeq, LIST or HiSeq, Genecore platform/EMBL) to achieve 20-30 million 75 base pair paired-end reads.

After quality filtering (>Q30), reads will be processed using the commercially available software CLC Genomics Workbench and mapped using the C. sativa cv. Santhica de novo transcriptome recently generated by the group at LIST-ERIN.

Then, DNA primers are designed to amplify the promoter of the identified genes, using as template hemp genomic DNA. DNA primers will be used to create the cassette to express, i.e. the bast-fibre promoter and the gene for surface-active protein, either hydrophobin or chaplin or rodlin or streptofactin.

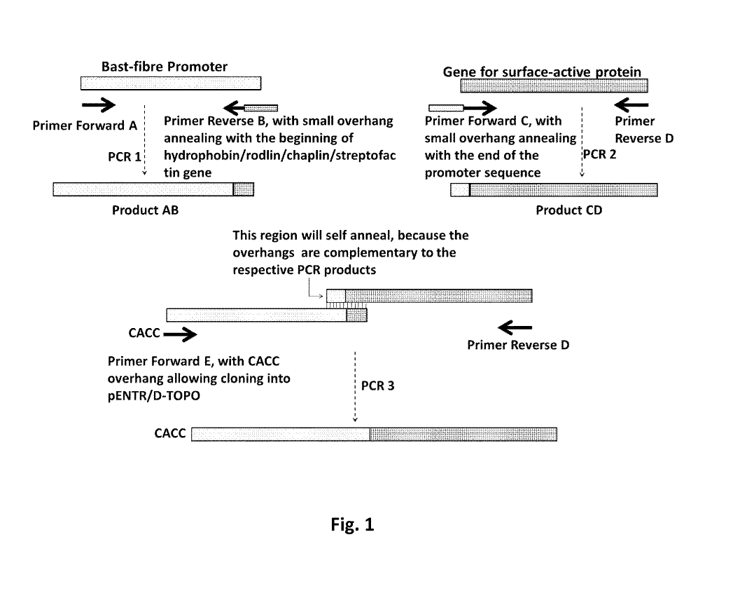

FIG. 1 indicates the PCR process between the bast-fibre promoter and the gene for surface-active protein.
As the bast-fibre promoter is a double-stranded DNA fragment, a forward primer A and a reverse primer B are needed for achieving the first PCR step. The reverse primer B will contain a small overhang adapted for annealing with the beginning of the gene for the surface-active protein. This first PCR step will yield the product AB.
Similarly, as the gene for the surface-active protein is a double-stranded DNA fragment, a forward primer C and a reverse primer D are needed for achieving the second PCR step. The forward primer C will contain a small overhang adapted for annealing with the end of the promoter sequence. This second PCR step will yield the product CD.
Then, in the course of the denaturation/annealing step inherent to a third PCR step, the product AB and the product CD will couple together. Those products have been indeed designed to self-anneal in the course of the PCR.
Then, the third PCR step on the double-stranded DNA fragment obtained by the coupling of AB and CD will be performed. A forward primer E, containing the small overhang with the sequence CACC adapted for cloning the cassette into the pENTR™/D-TOPO® vector will be used. A reverse primer D will be used for the complementary sequence.
This third PCR step will subsequently yield a cassette that is composed of the bast-fibre promoter fused to the gene coding for the surface-active protein.

This cassette will then be cloned in the pENTR™/D-TOPO® vector and recombined into the pEarleyGate 302 vector.

The pEarleyGate 302 plasmid is a binary vector that will replicate in both Escherichia co/i and Agrobacterium tumefaciens and has left border (LB) and right border (RB) sequences for Agrobacterium-mediated T-DNA transfer.

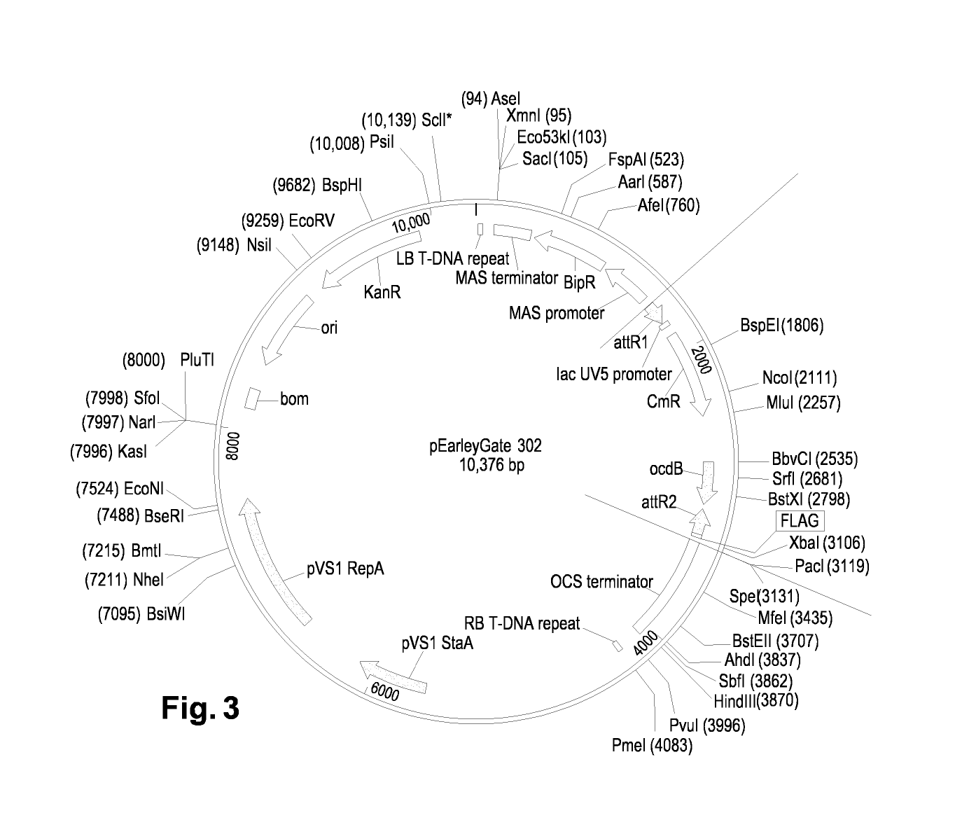

FIG. 3 depicts the pEarleyGate 302 plasmid. This vector is known in the literature (Earley K. W., et al., The Plant J., 2006, 45, 616-629).

The region comprised between attR1 and attR2 will be recombined with the pENTR/D-TOPO vector containing the bast-fibre promoter fused to the gene for the surface-active protein.

Hemp transformation will be performed via Agrobacterium tumefaciens GV3101 in accordance with the general knowledge (see notably US patent application published US 2012/0311744 A1). Both hypocotyl explants and calli will be tested for transformation (MacKinnon L., et al, Annual Report of the Scottish Crop Research Institute 2000/2001, eds. W. H. Macfarlane Smith and T. D. Heilbronn (SCRI, Invergowrie, Dundee), 2001, 84-86; Feeney M., et al, In Vitro Cell. Dev. Biol-Plant, 2003, 39, 578-585; Slusarkiewicz-Jarzina A., et al., Acta Biol. Cracov. Ser. Bot., 47, 2005, 145-151; Wang R., et al, Pak J. Bot., 2009, 41, 603-608; Lata H., et al, In vitro Cell Dev. Biol. Plant, 2009, 45, 12-19; Lata H., et al, Planta Med., 2010, 76, 1629-1633).

In the fungi and in the bacteria, the process of secretion of the surface-active protein is natural. However, in plants, such as fibre crops, this process is not natural. Therefore, to increase the chance of expressing those surface-active proteins into the fibre crops, notably the hemp, the genes can further be fused at the 5′ with a hemp β-expansin signal peptide (SEQ ID NO:1). This gene was previously identified thanks to the de novo assembly.

β-expansin is indeed a protein secreted by the plant cells that unlocks the network of wall polysaccharides, thus permitting turgor-driven cell enlargement (Cosgrove D. J., Nature, 2000, 407, 321-326).

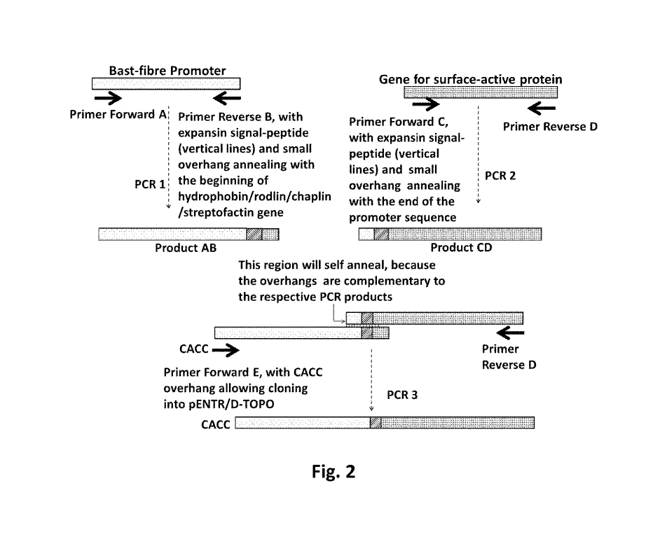

FIG. 2 indicates the PCR process between the bast-fibre promoter, the gene for surface-active protein and the gene for the β-expansin signal peptide.
As the bast-fibre promoter is a double-stranded DNA fragment, a forward primer A and a reverse primer B are needed for achieving the first PCR step. The reverse primer B will contain a small overhang adapted for annealing with the beginning of the gene for the surface-active protein. The reverse primer B will further contain the β-expansin signal peptide sequence.

This first PCR step will yield the product AB.
Similarly, as the gene for the surface-active protein is a double-stranded DNA fragment, a forward primer C and a reverse primer D are needed for achieving the second PCR step. The forward primer C will contain a small overhang adapted for annealing with the end of the promoter sequence. The forward primer C will further contain the β-expansin signal peptide sequence.

This second PCR step will yield the product CD.
Then, in the course of the denaturation/annealing step inherent to a third PCR step, the product AB and the product CD will couple together. Those products have been indeed designed to self-anneal in the course of the PCR.
Then, a third PCR step on the double-stranded DNA fragment obtained by the coupling of AB and CD will be performed. A forward primer E, containing the small overhang with the sequence CACC adapted for cloning the cassette into the pENTR™/D-TOPO® vector will be used. A reverse primer D will be used for the complementary sequence.
This third PCR step will subsequently yield a cassette that is composed of the bast-fibre promoter fused to the gene coding for the surface-active protein. This cassette will further contain the β-expansin signal peptide sequence.

This cassette will then be cloned in the pENTR™/D-TOPO® vector and recombined into the pEarleyGate 302 vector, depicted in FIG. 3.

The same process as it has been designed concerning the cassette devoid of the β-expansin signal peptide sequence will be undertaken in order to generate the genetically engineered plants.

1-15. (canceled)

16. A genetic engineering process of fibrous plant comprising bast fibres, said process comprising the following steps:
a) identification of the bast fibre promoter; and
b) amplification of the bast fibre promoter;
wherein the genetic engineering process further comprises the following step:
c) preparing a gene cassette by fusing the bast fibre promoter with at least one gene coding for a surface-active protein.

17. The genetic engineering process according to claim 16, wherein the step (c) further comprises the incorporation of the gene of the β-expansin signal peptide to the gene cassette.

18. The genetic engineering process according to claim 17, wherein the β-expansin signal peptide is SEQ ID NO:1.

19. The genetic engineering process according to claim 16, wherein the process further comprises the following step:
cloning the gene cassette in a first vector.

20. The genetic engineering process according to claim 19, wherein the process further comprises the following step
recombining the first vector into a second vector.

21. The genetic engineering process according to claim 19, wherein the first vector is pENTR™/D-TOPO® vector.

22. The genetic engineering process according to claim 20, wherein the second vector is pEarleyGate 302 vector.

23. The genetic engineering process according to claim 16, wherein the step (a) is performed by formation of complementary deoxyribonucleic acid libraries, cDNA libraries, and subsequent high-throughput sequencing, RNA-Seq.

24. The genetic engineering process according to claim 23, wherein the formation and sequencing are performed by using Illumina® Sequencing Technology.

25. The genetic engineering process according to claim 16, wherein the step (a) is performed in at least one of the above snap-point, ASP, part or in the below snap-point, BSP, part of the stem of the fibrous plant.

26. The genetic engineering process according to claim 16, wherein the step (b) is performed by means of polymerase chain reaction, PCR.

27. The genetic engineering process according to claim 16, wherein the surface-active protein is selected from the group of hydrophobins, chaplins, rodlins, and streptofactins.

28. The genetic engineering process according to claim 16, wherein the fibrous plant is selected from the group of flax, hemp, jute, kenaf, ramie, sisal, cotton and nettle.

29. The genetic engineering process according to claim 16, wherein the process is performed via Agrobacterium tumefaciens GV3101.

30. A fibrous plant obtained by a genetic engineering process of fibrous plant comprising bast fibres, comprising the following steps:
a) identification of the bast fibre promoter; and
b) amplification of the bast fibre promoter;
wherein the genetic engineering process further comprises the step of preparing a gene cassette by fusing the bast fibre promoter with at least one gene coding for a surface-active protein,
wherein the fibrous plant comprises surface-active proteins.

31. The fibrous plant according to claim 30, wherein the surface-active proteins are selected from the group of hydrophobins, chaplins, rodlins and streptofactins.

32. The fibrous plant according to claim 30, wherein the fibrous plant is selected from the group of flax, hemp, jute, kenaf, ramie, sisal, cotton and nettle.

33. The fibrous plant according to claim 30, wherein the surface of the fibrous plant is hydrophobic.
